(19) United States
(12) Patent Application Publication
(10) Pub. No.: US 2016/0174982 A1
(43) Pub. Date: Jun. 23, 2016

(54) APPARATUS AND METHOD FOR ASSISTING WITH WOUND CLOSURE

(71) Applicant: THE CLEVELAND CLINIC FOUNDATION, Cleveland, OH (US)

(72) Inventors: Marat Yanavitski, Beachwood, OH (US); Shengqiang Gao, Beachwood, OH (US); Eugene Jung, Beachwood, OH (US); William Kolosi, Cleveland, OH (US)

(21) Appl. No.: 14/976,450

(22) Filed: Dec. 21, 2015

Related U.S. Application Data
(60) Provisional application No. 62/095,324, filed on Dec. 22, 2014.

(51) Int. Cl. A61B 17/08

(52) U.S. Cl. CPC A61B 17/085; A61B 2017/086

(57) ABSTRACT

An apparatus for assisting with wound closure includes oppositely disposed, and oppositely facing, proximal and distal surfaces separated by an apparatus body. The distal surface is configured for selective attachment to a patient tissue surface. At least one of the proximal surface, the distal surface, and the apparatus body is made substantially from an polymeric material. The polymeric material includes a structurally incorporated ability to self-repair damage caused by mechanical usage over time. A method of assisting with wound closure is also provided.

### RELATED APPLICATION

This application claims priority from U.S. Provisional Application No. 62/095,324, filed 22 Dec. 2014, the subject matter of which is incorporated herein by reference in its entirety.

### TECHNICAL FIELD

This disclosure relates to an apparatus and method for use of a wound closure assistance apparatus and, more particularly, to a wound closure assistance apparatus and method that is placed upon the patient tissue and assists with reapproximating the tissue after a surgical procedure.

### BACKGROUND

Access site complications (bleeds, arteriovenous fistula formation, and dissections), which are some of the possibly serious and costly complications of percutaneous intervention and structural interventions, may add to the cost, mortality, and morbidity of procedures. The current methodology is to close an access site surgical wound after the procedure is finished, or to use one or more suture-based devices to pre-close the arteriotomy or other surgical wound. Limitations of the latter, pre-closure method include operator-dependence and a limited timeframe for availability (i.e., can only pre-close in the cath lab, so indwelling devices, such as Impella or ECMO cannulae, cannot be preclosed).

### SUMMARY

In an aspect, an apparatus for assisting with wound closure is provided. The apparatus comprises oppositely disposed, and oppositely facing, proximal and distal surfaces separated by an apparatus body. The distal surface is configured for selective attachment to a patient tissue surface. At least one of the proximal surface, the distal surface, and the apparatus body is made substantially from an polymeric material. The polymeric material includes a structurally incorporated ability to self-repair damage caused by mechanical usage over time.

In an aspect, a method of assisting with wound closure is provided. An apparatus including oppositely disposed, and oppositely facing, proximal and distal surfaces separated by an apparatus body is provided. The apparatus is at least partially made from an polymeric material having a structurally incorporated ability to self-repair damage caused by mechanical usage over time. The distal surface is selectively attached to a patient tissue surface. A surgical wound is created through an entire thickness of the apparatus body and into the attached patient tissue. The surgical wound is maintained open via at least one of interposition of a surgical item through the surgical wound and application of tension to the patient tissue on laterally opposite sides of the surgical wound. With the apparatus attached to the patient tissue, the surgical item is removed. With the apparatus attached to the patient tissue, the polymeric material is permitted to self-repair the surgical wound through the apparatus body by reducing a size of the surgical wound.

### BRIEF DESCRIPTION OF THE DRAWINGS

For a better understanding, reference may be made to the accompanying drawings, in which:

FIG. 1 is a top view of an apparatus according to an aspect of the disclosure;

FIG. 2 is a cross-sectional view taken along line 2-2 of FIG. 1;

FIG. 3 is a perspective view of the apparatus of FIG. 1;

FIGS. 4A-4D depict an example sequence of operation of the apparatus of FIG. 1

FIGS. 5A-5C are schematic top views of example configurations of the apparatus of FIG. 1;

FIGS. 6A-6C are schematic top views of example assembly components of the apparatus of FIG. 1;

FIGS. 7A-7C are schematic top views of example assembly components of the apparatus of FIG. 1;

FIG. 8A is a schematic top view of an example configuration of the apparatus of FIG. 1; and

FIG. 8B is a cross-sectional view taken along line B-B of FIG. 8A.

### DESCRIPTION OF ASPECTS OF THE DISCLOSURE

Unless defined otherwise, all technical and scientific terms used herein have the same meaning as is commonly understood by one of skill in the art to which the present disclosure pertains.

As used herein, the term “subject” can be used interchangeably with the term “patient” and refer to any warm-blooded organism including, but not limited to, human beings, pigs, rats, mice, dogs, goats, sheep, horses, monkeys, apes, rabbits, cattle, farm animals, livestock, etc.

As used herein, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” and “between about X and Y” can be interpreted to include X and Y.

As used herein, phrases such as “between about X and Y” can mean “between about X and about Y.”

As used herein, phrases such as “from about X to Y” can mean “from about X to about Y.”

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “directly adjacent” another feature may have portions that overlap or underlie the adjacent feature, whereas a structure or feature that is disposed “adjacent” another feature may not have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms can encompass different orientations of a device in use or operation, in addition to the orientation depicted in the figures. For example, if a device in the figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

The invention comprises, consists of, or consists essentially of the following features, in any combination.

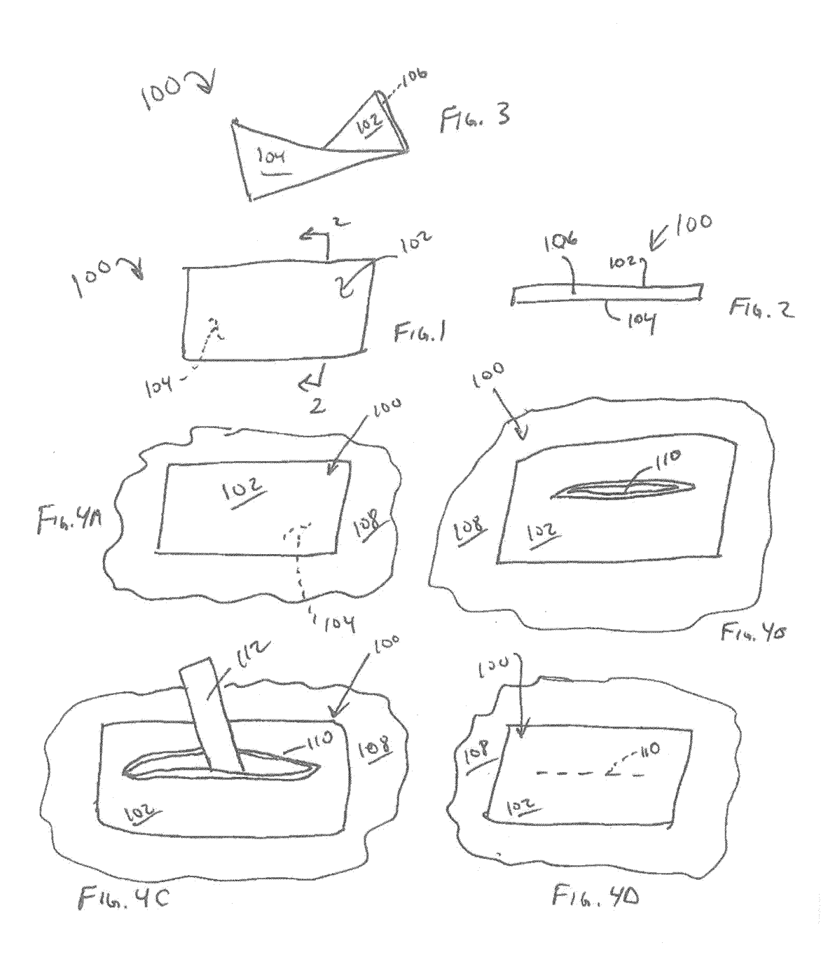

FIGS. 1-3 depict an apparatus 100 for assisting with wound closure. The apparatus 100 includes an polymeric material (such as, but not limited to, silicone) having oppositely disposed, and oppositely facing, proximal and distal surfaces 102 and 104, respectively, longitudinally separated by a apparatus body 106. The apparatus 100 is shown in the Figures as being a substantially planar, tissuelike construct, being relatively two-dimensional in nature and having proximal and distal surfaces 102 and 104 which are much larger than a thickness of the apparatus 100 taken between those surfaces (i.e., a thickness of the apparatus body 106). The distal surface 104 is configured for selective attachment to a patient tissue surface (not shown, in FIG. 1). The apparatus 100 is generally, though not exclusively, intended for use before a surgical wound is made, as a pre-treatment.

The apparatus 100 may be made at least partially from a material which is bioresorbable, bio-absorbable, and/or non-degrading and biocompatible, and may be relatively flexible, as shown in FIG. 3. For example, at least one of the proximal surface 102, the distal surface 104, and the apparatus body 106 may be made substantially from an polymeric material. The apparatus 100 may substantially conform in vivo to a particular patient tissue (e.g., when the apparatus 100 is flexible). Additionally or alternatively, the apparatus 100 may be physically configured for a particular patient tissue (e.g. contoured or cut-out to fit) before installation. Examples of suitable polymeric materials for at least a portion of the apparatus 100 include elastomers; liquids, gels or paste that can form a layer of plastic layer in situ, such as by crosslinking triggered by UV light, moisture, or any other suitable actuator; and non-elastomers, such as, but not limited to, polylactic acid (“PLA”), poly-l-lactide acid (“PLLA”), poly(lactic-co-glycolic) acid (“PLGA”), polyurethane, or the like.

At least a portion of the material forming the apparatus 100 may be applied and/or cured in situ to a patient tissue for assistance with a surgical wound in and/or around a blood vessel or any other patient tissue through which access, in the form of a surgical wound, is desired. Though depicted as a standalone sheet in the Figures for ease of description, the apparatus 100 could be provided to the patient tissue as a liquid, gel, paste, sheet, bracelet, tape, or the like, and may be applied to the patient tissue via any suitable device or applicator.

Any suitable device, substance, and/or applicator could also or instead be used to chemically, mechanically, and/or adhesively attach part or all of a surface of the apparatus 100 to the patient tissue. While many use environments of the apparatus 100 will include direct attachment of the apparatus 100 to the patient tissue, it is also contemplated that some intervening member (e.g., an adhesive sheet or other adhesive component, not shown, for selectively attaching the distal surface 104 to the patient tissue surface) may be interposed between the apparatus 100 and the patient tissue.

The apparatus 100 may be made at least partially from a self-healing polymer, which is characterized herein as an elastomeric material including a structurally incorporated ability to self-repair damage caused by mechanical usage over time. Any suitable “self-healing” material may be provided for use with the apparatus 100. The apparatus 100 may additionally or alternatively be made from an at least partially elastic material, which is capable of substantially regaining an initial configuration after a temporary mechanical alteration or damage, even if the actual structure of the material retains some “memory” or artifact of that alteration or damage. The term “self-repair” is used herein to indicate, without preference, either or both of these two types of material for a particular situation.

The apparatus 100 may also include a “valve” like feature (not shown). For example, the apparatus 100 could include a valving mechanism and/or one or more “flaps” which coapt, much like heart valve leaflets, to selectively provide a fluid path through the apparatus body 106. During catheterization or other invasive procedures, surgical tools can go through the “valve” feature of the apparatus 10 and then penetrate the patient tissue. At the end of the procedure, upon removal of the surgical tools, the “valve” may close off access through the apparatus body 106, such as by flattening or coapting of flap(s) under fluid (e.g., blood) pressure from inside a patient tissue lumen.

Regardless of its exact configuration, the apparatus 100 may be used to assist with wound closure, such as via the example sequence of operation shown in FIGS. 4A-4D. An apparatus 100, similar to that shown in FIG. 1, is provided. At least a portion of the distal surface 104 of the apparatus 100 is selectively attached to a patient tissue 108 surface, as shown in FIG. 4A, such as via an adhesive interposed therebetween. The patient tissue 108 surface may be, for example, the external surface of a blood vessel (beneath the skin), but could be any suitable patient tissue 108. The attachment can be made in any suitable manner, including, but not limited to, permanent or temporary adhesive components, suturing, stapling, wiring (e.g., metal wiring), or any other desired attachment scheme.

As shown in FIG. 4B, a surgical wound 110 is created (e.g., via a scalpel cut) through an entire thickness of the apparatus body 106 and into the attached patient tissue 108. For example, with the distal surface 104 attached (directly or indirectly) to the patient tissue 108, the user can create a surgical wound 110 through an entire thickness of the apparatus body 106 and into the attached patient tissue 108. It should be noted that the surgical wound 110 could be created before, during, and/or after attachment of the apparatus 100 to the patient tissue 108.

The surgical wound 110 is then held open, such as via interposition of a surgical item 112 through the surgical wound 110 as shown in FIG. 4C. Though not shown in the Figures, a retractor, forceps, surgeon's gloved finger, trocar, catheter, any other instrument/tool, or the like may be interposed through the surgical wound 110 and into the depth of the patient tissue 108 as a surgical item 112, and/or gentle “spreading” lateral tension could be applied to the apparatus 100 and/or the patient tissue 108 on laterally opposite sides of the surgical wound 108 to hold the surgical wound 110 open without the presence of an intervening surgical item 112.

When access to the patient tissue 108 through the surgical wound 110 is no longer desired by the user, the surgical item 112 is removed from the surgical wound 110, and/or any lateral tension applied to the surgical wound 110 is ceased. As shown in FIG. 4D, then, the apparatus 100 (e.g. an elastomeric material thereof) is permitted to self-repair the surgical wound 110 formerly extending through the apparatus body 106 by reducing a size of the surgical wound 110. The (former) surgical wound 110 is shown as a dashed line in FIG. 4D. However, depending upon the self-repairing properties of the material(s) of the apparatus 100, there might be no visible indication left of the (former) position of the surgical wound 110 once the apparatus 100 has suitably self-repaired.

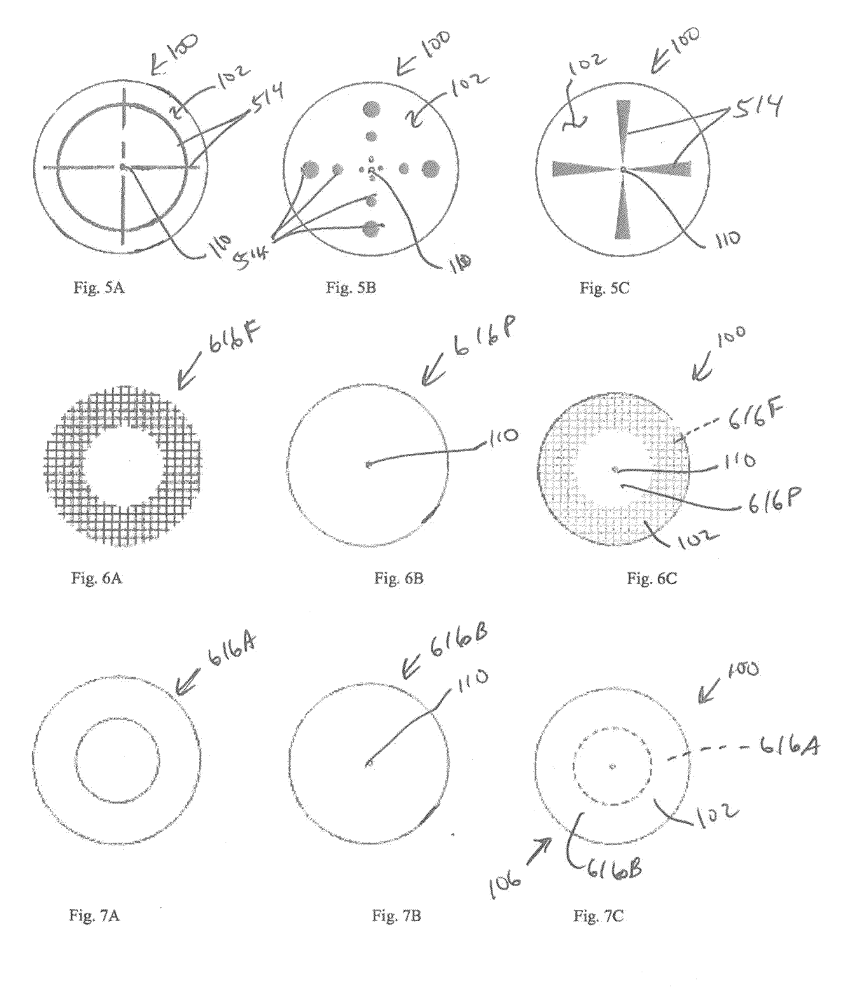

FIGS. 5A-5C depict example configurations of an apparatus 100 having at least one user-perceptible marker 514 (e.g., a fiducial marker) conveying information to a user via the position of the marker 514 with respect to the apparatus body 106. For example, the marker(s) 514 could be radiopaque, radiolucent, visible to a user's naked eye (optionally under ultraviolet or infrared light), or otherwise configured for detection and perception by the user, visually and/or with the assistance of imaging technologies (e.g., ultrasound, fluoroscopy, or any others) which are available to the user.

The marker(s) 514 may be encapsulated within the apparatus body 106, particularly if the marker 514 material is potentially irritating to the patient or undesirably protruding from the proximal surface 102. The marker(s) 514 may also or instead be directly attached to a chosen one of the proximal surface 102 and the distal surface 104.

As shown in the example configurations of FIGS. 5A-5C, the markers 514 may take any suitable shape, configuration, and placement, and may be provided for any desired purpose. For example, the rotationally symmetrical patterns of markers in FIGS. 5A-5C are centered about a “point” or “puncture” type surgical wound 110, and may provide a “bullseye” target for a user to relatively precisely initially locate, or later relocate (particularly some time after the surgical procedure) the site of the desired, existing, or former surgical wound 110. For example, any of the patterns of markers 514 shown in FIGS. 5A-5C, or any other patterns/locations of markers 514 (e.g., a simple line), could be used to facilitate the creation of an incision-type surgical wound 110 and/or the insertion of a catheter through the apparatus 100 in an over-the-wire delivery technique.

As another example configuration for the apparatus 100, and as shown in FIGS. 6A-8B, the apparatus body 106 may be comprised of a plurality of stacked body layers 616, for any reason and in any configuration(s). FIG. 6A depicts a fabric “ring” body layer 616F which is bonded to an elastomeric or other type of polymer “patch” body layer 616P to form the multi-body-layer 616 apparatus 100 shown in FIG. 6C. The fabric body layer 616F may be bonded to the patient tissue 108 with an adhesive, and the fabric body layer 616F may be bonded to (e.g., molded into) the polymer body layer 616P. When present, this fabric-polymer body layer 616 configuration may be helpful in resisting unwanted stretching of the polymer of the apparatus body 106 and/or in adhering the polymer body layer 616P to the patient tissue 108.

While the “ring” body layer in FIGS. 6A-6C is described as being “fabric” and the “patch” body layer of those Figures as “polymer”, it is contemplated that any suitable material(s) could be used for the various body layers 616 of the device. In other words, at least a portion of the proximal surface 102 may be made of a first material, and a majority of the distal surface 104 may be made from a second material, different from the first material.

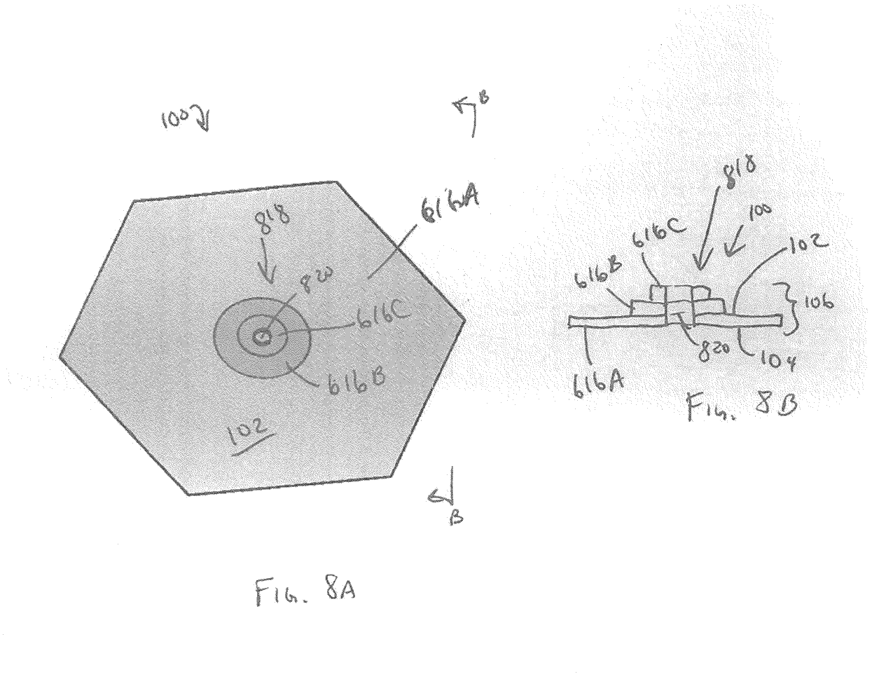

As another example, FIGS. 7A-7C depict the components and construction of an apparatus 100 having “ring” and “patch” body layers that are made of the same material. The “ring” body layer 616A and the “patch” body layer 616B may have the same or different thicknesses, and the aggregation of the body layers 616A and 616B into the apparatus body 106 may be used to provide desired mechanical properties (e.g., stiffness, flexibility, thickness, rigidity, or any other mechanical properties) to the apparatus 100, for any desired purpose. For example, it may be desirable for the outer “rim” of the apparatus 100 to be stiffer than the center portion, for ease of manipulation.

Turning to FIGS. 8A-8B, an apparatus 100 including a coupling feature 818 configured to selectively attach to an installation device (not shown) is provided. The apparatus body 106 of FIGS. 8A-8B is comprised of multiple body layers 616A, 616B, 616C, which are located in a central portion of the apparatus 100. The body layers 616A, 616B, and 616C are stacked to provide a reinforced center portion serving as a coupling feature 818. The coupling feature 818 may include a center aperture 820 which is configured to interact with a guidewire, insertion catheter, grasper, or other installation device to maintain the apparatus 100 in desired position(s) during transport to the patient tissue 108 and deployment there.

During use of an apparatus 100 having a coupling feature 818, the apparatus 100 may be selectively attached to an installation device via the coupling feature 818. The apparatus 100 is then manipulated into a predetermined installation position with respect to the patient tissue 108 via the installation device. The coupling feature 818 may then be released from the installation device at the predetermined installation position.

Self-repairing of the apparatus 100 may have the effect of bringing the edges of the surgical wound 110 made in the patient tissue 108 into close enough proximity to achieve and/or facilitate closure. For example, if the self-repairing properties of the apparatus 100 are sufficiently strong, the apparatus 100 may be able to achieve approximation which presses together the wound edges of the underlying patient tissue 108, achieving closure and healing without any additional interaction. Another example is that, by simply bringing the patient tissue 108 wound edges into close enough proximity (even if not under approximation pressure or even necessarily touching), wound closure can be achieved through scar tissue formation between the wound edges without an additional closure device being provided.

A third example includes, in the case of large-bore device procedures, the apparatus 100 drawing the wound edges of the patient tissue 108 back to a size compatible with a commercially available closure device—or even sutures—designed for small-bore incisions, to minimize the size of the closure device used. In this third example, the apparatus 100 could include features to assist with the installation and/or retention of one or more closure devices.

It is contemplated that the apparatus 100 could be applied, additionally or alternatively, to patient tissue 108 incorporating an existing at-least-partial surgical wound 110, or even to an existing accidental/traumatic wound (not shown), particularly in situations where medical considerations/strategies make creation of the surgical wound 110 before application of the apparatus 100 to the patient tissue 108 desirable.

It is also contemplated that some additional material (e.g., an adhesive such as, but not limited to, a glue or sticky gel) may be provided to any form of self-repairing material for the apparatus 100 to assist with the desired wound closure.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials; however, the chosen material(s) should be biocompatible for many applications. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. A “predetermined” status may be determined at any time before the structures being manipulated actually reach that status, the “predetermination” being made as late as immediately before the structure achieves the predetermined status. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Other aspects, objects, and advantages can be obtained from a study of the drawings, the disclosure, and the appended claims.

We claim:

1. An apparatus for assisting with wound closure, the apparatus comprising:
oppositely disposed, and oppositely facing, proximal and distal surfaces separated by an apparatus body, the distal surface being configured for selective attachment to a patient tissue surface;
wherein at least one of the proximal surface, the distal surface, and the apparatus body is made substantially from an polymeric material; and
wherein the polymeric material includes a structurally incorporated ability to self-repair damage caused by mechanical usage over time.

2. The apparatus of claim 1, wherein the polymeric material is bio-absorbable.

3. The apparatus of claim 1, wherein the polymeric material is non-degrading and biocompatible.

4. The apparatus of claim 1, including an adhesive component for selectively attaching the distal surface to the patient tissue surface.

5. The apparatus of claim 1, including at least one of a suturing, stapling, and wiring component for selectively attaching the distal surface to the patient tissue surface.

6. The apparatus of claim 1, including at least one user-perceptible marker conveying information to a user via the position of the marker with respect to the apparatus body.

7. The apparatus of claim 6, wherein the marker is encapsulated within the apparatus body.

8. The apparatus of claim 6, wherein the marker is directly attached to the proximal surface.

9. The apparatus of claim 1, wherein the apparatus body is comprised of a plurality of stacked body layers.

10. The apparatus of claim 9, wherein at least a portion of the proximal surface is made of a first material, and a majority of the distal surface is made from a second material, different from the first material.

11. The apparatus of claim 1, including a coupling feature configured to selectively attach to an installation device.

12. A method of assisting with wound closure, the method comprising:
providing an apparatus including oppositely disposed, and oppositely facing, proximal and distal surfaces separated by an apparatus body, the apparatus being at least partially made from an polymeric material having a structurally incorporated ability to self-repair damage caused by mechanical usage over time;
selectively attaching the distal surface to a patient tissue surface;
creating a surgical wound through an entire thickness of the apparatus body and into the attached patient tissue;
maintaining the surgical wound open via at least one of interposition of a surgical item through the surgical wound and application of tension to the patient tissue on laterally opposite sides of the surgical wound;
with the apparatus attached to the patient tissue, removing the surgical item; and
with the apparatus attached to the patient tissue, permitting the polymeric material to self-repair the surgical wound through the apparatus body by reducing a size of the surgical wound.

13. The method of claim 12, wherein creating a surgical wound includes:
with the distal surface attached to the patient tissue, creating a surgical wound through an entire thickness of the apparatus body and into the attached patient tissue.

14. The method of claim 12, wherein selectively attaching the distal surface to a patient tissue surface includes providing an adhesive component and selectively attaching the distal surface to the patient tissue surface with the adhesive component.

15. The method of claim 12, wherein selectively attaching the distal surface to a patient tissue surface includes providing at least one of a suturing, stapling, and wiring component and selectively attaching the distal surface to the patient tissue surface with the at least one suturing, stapling, and wiring component.

16. The method of claim 11, including:
providing the apparatus with at least one user-perceptible marker; and
conveying information to a user via the position of the marker with respect to the apparatus body.

17. The method of claim 16, wherein providing the apparatus with at least one user-perceptible marker includes encapsulating the marker within the apparatus body.

18. The method of claim 16, wherein providing the apparatus with at least one user-perceptible marker includes directly attaching the marker to the proximal surface.

19. The method of claim 12, wherein providing the apparatus includes providing an apparatus body comprised of a plurality of stacked body layers.

20. The method of claim 12, wherein providing the apparatus includes providing the apparatus with a coupling feature, the method including:
selectively attaching the apparatus to an installation device via the coupling feature;
manipulating the apparatus into a predetermined installation position with respect to the patient tissue via the installation device; and
releasing the coupling feature from the installation device at the predetermined installation position.
